(19) United States
(12) Patent Application Publication
(10) Pub. No.: US 2026/0257204 A1
(43) Pub. Date: Sep. 3, 2026

(54) SURFACE-CATALYTICALLY FINISHED POLYMER FIBRES AND/OR POLYMERIC SHEETS, METHOD FOR SURFACE-CATALYTICALLY FINISHING SAME, AND USE THEREOF FOR PRODUCING AN AIR PURIFICATION FILTER

(71) Applicant: HOCHSCHULE NIEDERRHEIN, Krefeld (DE)

(72) Inventors: Robert GROTEN, Wegberg (DE); Andreas ROPPERTZ, Krefeld (DE); Isabelle HEINST, Billerbeck (DE); Marcel SLADKOV, Kamp-Lintfort (DE)

(21) Appl. No.: 18/879,939

(22) PCT Filed: Jul. 4, 2023
(86) PCT No.: PCT/EP2023/068364

§ 371 Date: Dec. 30, 2024

(30) Foreign Application Priority Data
Jul. 4, 2022 (DE) 10 2022 116 641.4

(51) Int. Cl. B01J 23/42; B01D 39/16; B01D 46/00; B01D 53/86; B01J 21/06; B01J 31/06; B01J 31/38; B01J 35/40; B01J 35/58; B01J 37/02

(52) U.S. Cl. CPC B01J 23/42; B01D 39/1623; B01D 46/0001; B01D 46/0027; B01D 53/864; B01J 21/063; B01J 31/06; B01J 31/38; B01J 35/40; B01J 35/58; B01J 37/0215; B01D 2239/0407; B01D 2239/0492; B01D 2239/0618; B01D 2239/10; B01D 2255/1021; B01D 2255/20707; B01D 2255/915; B01D 2255/9202; B01D 2255/9205; B01D 2257/502; B01D 2258/02

(57) ABSTRACT

The present disclosure relates to a method for surface-catalytically finishing polymer fibres and/or polymeric sheets, said method comprising the steps: a) providing a polymer fibre or a polymeric sheet; b) providing an aqueous dispersion of a catalyst support particle; and c) contacting the polymer fibre or the polymeric sheet with the aqueous dispersion of the catalyst support particle, the method being characterised in that the catalyst support particle has a particle size in a range of between at least 0.5 μm and at most 20 μm, and in that the contacting in step c) takes place at a temperature in a range of between at least 15° C. and at most 60° C. above the glass transition temperature T_{G} of the polyester, and wherein the catalyst support particle is impregnated with a catalytically active metal or metal oxide. The disclosure also relates to: a polyester fibre or polyester sheet with a corresponding finish; and the use of same for producing a filter for particle separation in an air purification system.

### CROSS-REFERENCE TO RELATED APPLICATIONS

This application is a U.S. National Phase Application under 35 U.S.C. 371 of International Application No. PCT/EP2023/068364, filed on Jul. 4, 2023, which claims the benefit of German Patent Application No. 10 2022 116 641.4, filed on Jul. 4, 2022. The entire disclosure of the aforementioned German Patent Application is incorporated herein by reference.

### FIELD

The present disclosure relates to a method for surface-catalytically finishing polymer fibres and/or polymeric sheets with catalyst particles, and the use of the fibres and/or sheets thus finished.

### BACKGROUND

This section provides background information related to the present disclosure which is not necessarily prior art.

Heterogeneous catalysis is a reaction of gaseous or liquid substances on the surface of a solid partner, the catalyst. The presence of a catalyst increases the reaction speed or reduces the activation energy. The externally measurable reaction speed r_{eff} is dependent on several influencing factors, e.g. the phase boundary, the bulk density of the catalyst, and the pore structure. Selection of the catalyst is based on the desired reactions to be catalysed. Catalysts used in the case of, for example, gas purification processes are usually metals or metal compounds, as well as their oxides. These metals are applied to an otherwise inactive support material with a large external and internal surface area. A catalyst should have high activity and selectivity with regard to the reactions to be achieved. A second requirement is high stability, i.e. resistance to mechanical influences, such as abrasion, and chemical resistance. For example, platinum may be used as catalyst for the oxidative conversion of CO to CO₂. Here, conversion of toxic carbon monoxide into the less harmful carbon dioxide takes place on a platinum surface, which catalyses the reaction. For this end, oxygen and carbon monoxide molecules first bind to the surface of the platinum before they react to form carbon dioxide and simultaneously detach from the platinum, which remains unchanged during this process. About 99 percent of the platinum surface is comprised of smooth surfaces, while just over one percent is comprised of steps between the individual smooth layers of platinum atoms. Both types of surfaces are catalytically active, wherein the steps exhibit a stronger catalytic activity than the smooth surfaces.

For an effective heterogeneous catalytic conversion of gases in particular, a large phase boundary between the gas to be converted and the catalyst is necessary. For this purpose, catalytic finishing of fibres or sheets is suitable.

A practical example of an application of such a heterogeneous gas catalysis is the use of fine dust filters such as hose filters during the pneumatic conveying of organic dusts for product separation. However, a side effect of these processes is that, at elevated temperatures, the formation of fire nests may occur, in which harmful gases such as carbon monoxide are formed. The resulting carbon monoxide must be oxidized to carbon dioxide before it is allowed to be released into the exhaust air. Compliance with corresponding carbon monoxide limits will be a problem for industrial applications in the future. In particular, relatively low temperatures are possible for catalytic exhaust air purification.

Here, a problem is the so-called fixing of the catalyst to the fabric or fibre surface. Such fixing is usually carried out by means of a binder, such as an adhesive. However, such a fixing of the catalyst or catalytically active particles often does not meet the mechanical requirements for corresponding sheets or fibres, so that the applied catalyst particles are abraded from the surface and the catalytic activity of the sheets or fibres decreases significantly accordingly. Also, the binder occupies a not insignificant part of the catalytically active surface.

Furthermore, with a corresponding surface-catalytically finished fabric, knit, or nonwoven, the flow resistance should preferably remain unchanged compared to a not surface-catalytically finished fabric, knit, or nonwoven.

The document EP 1738823 A1 discloses a catalytically active unit with a support material, wherein the catalytically active unit or the support material comprises polymer particles, in particular polymeric nanoparticles, and/or wherein the support material has been applied with polymer particles, in particular polymeric nanoparticles, the polymer particles comprising at least one catalytically active component. The catalytically active unit according to the present disclosure is particularly suitable for the removal of harmful substances, odorants, and toxins of all kinds, in particular from air and/or gas streams, and for protection against chemical toxins, in particular warfare agents, for example in CBRN-protective materials (e.g. protective clothing).

JP 2004024937 A discloses a device for producing a catalytic hose filter with a container for a catalytic slurry, a circulation pump for the catalytic slurry in the container for the catalytic slurry, a viscosity sensor for the catalytic slurry in the container for the catalytic slurry, a feed device for the catalytic slurry, whose operation is controlled by measured values from the viscosity sensor, a flat base on which the bag filter is placed, a press plate to press the bag filter onto a surface of the flat base and impregnate the bag filter with the catalytic slurry, a drive device for the press plate, and a pair of squeezing rollers installed at an upper part of the container for the catalytic slurry.

The document RU 2399391 C1 discloses a filter-catalyst composite material for purification of air from aerosols and carbon monoxide which contains two layers of a fibrous filter medium and one layer of catalytic material. The layer of catalytic material consists of needle felt with a surface density of 220 to 250 g/m³ filled with a finely ground palladium-containing low-temperature carbon monoxide oxidation catalyst with a particle size of 100 nm in the following weight ratio: catalyst: 15 to 50%; fibrous filter medium: the remainder, and is arranged between the layers of filter medium made from electrostatic fine-threaded polymer fibres.

US 2021 069621 A1 discloses a filter that can remove particles with a size of 2.5 μm and/or other airborne harmful air substances, wherein the filter has fibres with an average diameter of no more than 500 nm, wherein the fibres consist of at least 90 wt. % polyacrylonitrile, based on all fibres in the filter; and a catalyst with at least 90 wt. % TiO₂, based on all catalytic metals in the filter, which is dispersed on the fibres. The fibres do not need to be loaded. The TiO₂ may be condensed or precipitated from a liquid containing the TiO₂ and the fibres using simple methods. The catalyst may be activated by UV irradiation to decompose particles with an average particle size of 2.5 μm or less and/or other harmful substances in the air. Such filters may be used near areas with vehicle traffic, e.g. as elements of traffic lights, and may be used for controlled purification of polluted air.

US 2009235625 A1 discloses a filter comprising an air-permeable membrane with pores. A nanoparticle precursor is dispersed in the pores, and the nanoparticle precursor reacts to a stimulus to form a catalytically active nanoparticle. A corresponding method is also provided.

BE 883343 A discloses a textile material containing polyester fibres with excellent opacity and tactile properties, wherein the material is characterised in that it comprises a substrate containing polyester fibres, wherein the fibres have been finished, up to about 20 wt. % of the textile material, with titanium dioxide particles with an average particle size of at least about 0.18 micron, wherein the durability of the bonding of the particles to the textile fibres is such that at least 50% of the particles remain fixed to the surface of the textile fibres after five AATCC standard washes.

JP 2009191369 A1 discloses that a coating film made of a diallyl-dimethylammonium salt polymer is formed on the surface of a polyester fibre that forms a curtain fabric, and porous fine particles carrying a metal oxide catalyst and/or a metal catalyst are adsorbed on the coating film.

US 2002197396 A1 discloses a method and an apparatus for treating yarn to improve performance characteristics, such as e.g. odor adsorption capacity, of the yarn while maintaining the physical properties associated with the yarn, such as e.g. feel and grip. In the methods for treating the yarn, solid particles such as activated carbon are incorporated into the yarn using an air dispersion technique or a batting technique. In the air dispersion technique, the solid particles are distributed over the yarn in a controlled stream of air. In the batting method, the solid particles are incorporated into the yarn while the yarn runs through a bath of solid particles. In the yarn treatment method, a binder may be applied to the yarn. The binder binds the solid particles to the yarn without affecting the performance characteristics of the solid particles. The yarn treatment method also includes curing the binder to permanently bond the solid particles to the yarn.

### SUMMARY

This section provides a general summary of the disclosure, and is not a comprehensive disclosure of its full scope or all of its features.

Therefore, it is an object of the present disclosure to provide a method for providing a surface-catalytically active polymer fibre or a polymeric sheet, wherein these have high mechanical stability and consistently good catalytic activity. According to a further aspect, it is an object of the disclosure to provide a catalytically active polyester sheet with high mechanical stability and consistently good catalytic activity, as well as a use of the same.

The disclosure proposes a method for surface-catalytically finishing polymer fibres and/or polymeric sheets, comprising the steps:
a) providing a polymer fibre or a polymeric sheet made of an amorphous or partially amorphous polymer;
b) providing an aqueous dispersion of a catalyst support particle;
c) contacting the polymer fibre or the polyester sheet with the aqueous dispersion of the catalyst support particle, wherein the catalyst support particle has a particle size in a range of between ≥0.5 μm and ≤20 μm, preferably between ≥1 μm and ≤10 μm, in particular between ≥1.5 μm and ≤5 μm, and the contacting in step c) takes place at a temperature in a range of between ≥15° C. and 60° C., preferably between ≥20° C. and ≤50° C., in particular between ≥30° C. and ≤40° C. above the glass transition temperature T_{G} of the polymer, and wherein the catalyst support particle is impregnated with a catalytically active metal or metal oxide.

A sheet within the meaning of the disclosure is a fabric, a knit, a nonwoven, a film, or a membrane.

Surface-catalytically finishing within the meaning of the disclosure is understood to be the application of a catalytically active substance to the surface of the polymer fibre or the polymeric sheet, so that fluids, such as gas or liquids, passing along the surface come into contact with the catalytically active substance and a conversion reaction of the fluid or a substance contained in the fluid may occur heterogeneously.

According to a preferred embodiment of the disclosure, the amorphous or partially amorphous polymer is a polyester or a polyamide, or a copolymer or blend of these. Polyesters within the meaning of the disclosure are in particular polyethylene terephthalate (PET), polybutylene terephthalate (PBT), polylactic acid (PLA), polytrimethylene terephthalate (PTT), polyethylene naphthalate (PEN), polycarbonate (PC), polyarylate (PAR), polyester carbonate (PEC), or copolymers or blends of these.

Surprisingly, it has been found that it is possible by means of the method according to the disclosure to bind catalyst support particles impregnated with a catalytically active metal or metal oxide in a mechanically stable manner to an amorphous or partially amorphous polymer. Mechanically stable within the meaning of the disclosure means that the catalyst support particles are washed off or mechanically abraded only to a very small extent. In particular, mechanically stable within the meaning of the disclosure means that, with a polymer fibre or a polymeric sheet finished according to the disclosure, ≥500 compressed air pulses with an overpressure of 6 bar, each lasting 1 second, lead to detachments of the catalytic particles of ≤5% based on the total mass of the catalytic particles. In this respect, the method according to the disclosure enables a dispersion fixation of catalytically active particles to a polymer fibre or a polymeric sheet. Here, it was surprisingly found that the catalyst support particles partially penetrate into the polymer structure and are thus connected in a mechanically stable manner to the polymer structure. The only partial penetration into the structure allows that a predominant part of the catalyst support particle surface and thus the catalytically active metals or metal oxides applied to the catalyst support particle are available for heterogeneous catalysis.

According to a preferred embodiment of the method according to the disclosure, the polymer fibre or the polymeric sheet is contacted at the specified temperature for a period of between ≥30 min and ≤360 min, preferably between ≥60 min and ≤240 min, in particular between ≥90 min and ≤120 min. It has been shown that such a contact period is sufficient to ensure a sufficient migration of the catalyst support particles impregnated with a catalytically active metal into the fibre structure or the sheet structure without causing the polymer to swell too much.

According to a preferred embodiment of the method according to the disclosure, the contacting of the polymer fibre or the polymeric sheet with the aqueous dispersion of a catalyst support particle takes place while a temperature gradient is provided. Here, it may preferably be provided that the polymer fibre or the polymeric sheet and the aqueous dispersion of a catalyst support particle are jointly heated from a lower temperature, such as room temperature, to the temperature to be provided in a range of between ≥15° C. and ≤60° C. above the glass transition temperature T_{G} of the polymer. Here, a heating rate in a range of between ≥1° C./min and ≤10° C./min, preferably between ≥2° C./min and ≤5° C./min, for example 3° C./min, may preferably be provided. After completion of a holding time of between ≥30 min and ≤360 min, preferably between ≥60 min and 240 min, in particular between ≥90 min and ≤120 min, cooling to a lower temperature may preferably occur. Here, the target temperature during cooling is preferably in a range of between ≥5° C. below the glass transition temperature T_{G} of the polymer and room temperature. During cooling, a cooling rate in a range of between ≥1° C./min and 20° C./min, preferably between ≥2° C./min and ≤10° C./min, for example 6° C./min, may preferably be provided. Here, it is preferably provided that the cooling rate is greater than the heating rate.

According to a further preferred embodiment of the method, the catalyst support particle is porous and preferably has a porosity Φ in a range of between ≥10% and 60%. Such a porosity advantageously increases the specific surface area of the catalyst support particle and thus the contact area available for heterogeneous catalysis, and also allows an improved fixation of the catalyst support particles to the polyester fibres or polymeric sheets. Porosity in the sense of this disclosure is 1 minus the quotient of the gross density of the catalyst support particle and the true density of the catalyst support particle's material according to the formula Φ=(1−ρ/ρ₀)*100%, wherein p corresponds to the gross density and po to the true density, and wherein ρ=m/(V_{solid}+Vₚₒᵣ) and ρ₀=m/V_{solid}.

According to a further embodiment of the method according to the disclosure, it is provided that the catalyst support particle is selected from the group consisting of titanium dioxide, cordierite, zeolites, magnesium silicate, or aluminium silicate. In the case of a magnesium silicate, talc is preferred. It has advantageously been shown that the mentioned compounds have good impregnability as well as chemical and mechanical stability.

According to a further embodiment of the method, it may be provided that the catalytically active metal is at least one metal selected from the group consisting of metals from the group of outer transition metals of the periodic table, preferably platinum, nickel, gold, silver, rhenium, cobalt, vanadium, chromium, copper, palladium, iridium, rhodium, and zirconium, or an oxide or mixed oxide thereof. Advantageously, the mentioned metals have good redox-catalytic properties, which may be advantageously used in the context of the application of polyester fibres or polyester sheets finished accordingly.

In an embodiment of the method according to the disclosure, the aqueous dispersion is adjusted to a pH value in a range of between ≥pH 1 and ≤pH 5. It has been shown that providing a correspondingly acidic environment facilitates the bonding of the catalyst support particles to the polymer structure. In particular, it may be provided that during the course of the method, i.e. during the period in which the polymer fibre or the polymeric sheet is contacted with the aqueous dispersion, a pH gradient is set. Here, it is particularly preferred that the pH value decreases during the time of contact. This may achieve a more uniform bonding and distribution of the catalyst support particles in the polymer structure.

According to a further embodiment of the disclosure, it may be provided that the aqueous dispersion of the catalyst support particle includes a dispersing agent. Preferably, the dispersing agent may be included in the dispersion in a concentration of between ≥0.01 g/L and ≤0.04 g/L based on the entire dispersion. According to a further preferred embodiment, for every 10 g of polymer fibre/polymeric sheet, between ≥50 mL and ≤250 mL, preferably between ≥80 mL and ≤150 mL, for example 100 mL, of dispersion is used.

According to another preferred embodiment of the disclosure, the dispersant may be an agent selected from the group consisting of XHT-S_SDB, SMS, _SDB by the company CHT Germany GmbH of Tubingen. Here, it may be provided that, to prepare a dispersion, a stock solution of a dispersant is first prepared, which is then added to the dispersion in a ratio of between ≤1:50 and ≥1:250, preferably between ≤1:80 and >1:150, for example 1:100.

According to another embodiment of the disclosure, it may be provided that the dispersion includes the catalyst support particle in a concentration in a range of between ≥5 g/L and ≤100 g/L, preferably between ≥15 g/L and 40 g/L, based on the entire dispersion. Such a concentration has proven to be particularly suitable to ensure a uniform coating of the catalyst support particle onto the polymer fibre or the polymeric sheet.

According to a preferred embodiment of the method, the polymer fibre or the polymeric sheet is loaded with between 5 wt. % and 20 wt. % of catalyst support particles. Here, the loading rate may be calculated from the quotient of the difference in weight of the polymer fibre or the polymeric sheet after the dispersion fixation of the catalyst support particles and the weight of the polymer fibre or the polymeric sheet before the dispersion fixation of the catalyst support particles, and the weight of the polymer fibre or the polymeric sheet before the dispersion fixation of the catalyst support particles.

According to an embodiment of the disclosure, it may be provided that the preferably used TiO₂ catalyst particles or other metallic or metal oxide catalyst support particles are impregnated, in the sense of a wet impregnation, with another metal salt or noble metal salt solution and thermally treated. The thermal treatment may include heating to a temperature of, for example, 400° C. with a high heating rate of up to 20 K/min. The impregnated catalyst support particle may be held at this temperature for a duration of 30 min and then cooled down in a defined manner with a cooling rate of, for example, 2 to 5 K/min.

With regard to the surface-catalytically finished polymer fibre or surface-catalytically finished polymeric sheet, the object of the disclosure is solved by the fibre or the sheet, which has catalyst support particles on its surface, wherein the catalyst support particles have, as a catalyst, at least one metal of the group of outer transition metals of the periodic system, preferably platinum, nickel, gold, silver, rhenium, cobalt, vanadium, chromium, copper, palladium, iridium, rhodium, and zirconium, or an oxide or a mixed oxide thereof, and wherein the catalyst support particles have at least partially penetrated into the polymer fibre or the polymeric sheet and are thus bonded to the polymer fibre or the polymeric sheet without any additional fastening means.

Surprisingly, it has been found that it is possible with the method according to the disclosure to fix catalyst support particles in a mechanically stable manner to the surface of a polymer fibre or a polymeric sheet without the need for a separate binder such as an adhesive. On the other hand, the catalyst support particles only penetrate into the polymer fibre or the polymeric sheet, or the surface thereof, to the extent that a predominant part of the particles protrudes from the surface and thus redox-active catalyst metals applied thereon come into sufficient contact with any gas passing along the surface of the polymer fibre or the polymeric sheet to be catalytically converted.

Surprisingly, it has been found that the catalyst support particles are mechanically immobilized on the surface of the fibre or the sheet in a manner so stable that ≥500 compressed air pulses with an overpressure of 6 bar, each lasting 1 second, lead to detachments of the catalytic particles of ≤5% based on the total mass of the catalytic particles.

With regard to the use, the disclosure proposes using the surface-catalytically finished polymer fibres or a corresponding surface-catalytically finished polymeric sheet according to the disclosure for the production of a filter for particle separation in an air purification system. In particular, the disclosure proposes using the surface-catalytically finished polymer fibres or a corresponding surface-catalytically finished polymeric sheet according to the disclosure for the production of a gas/solid filter for catalytic degradation of carbon monoxide.

For example, it is possible to provide a hose filter for a particle separator for the separation of organic dusts from a gas stream, which is able to catalytically convert any carbon monoxide (CO) formed in the separated organic dusts or the formed filter cake into carbon dioxide (CO₂), thus removing it from the filtered gas stream. This may overcome a problem in mechanical exhaust gas purification which occurs when, for example, fire or ember nests form in the separated filter cake and CO is formed during the thermal decomposition of the filter dusts. Such CO formation, however, is undesirable, as the filtered gas stream is contaminated by this CO, and regulatory requirements in regard to the exhaust gas stream may not be able to be met. Due to the catalytically finished polymer fibres or polymeric sheets according to the disclosure, relatively low temperatures for catalytic exhaust air purification are feasible. Another advantage of the use of the surface-catalytically finished polymer fibres or polymeric sheets according to the disclosure is that the catalytically active substance bonds to the fibres or the sheet in a mechanically stable manner, so that a removal of the catalytically active substance through mechanical loads, such as one which may occur in filter systems during operation and especially during cleaning by counterflow, can be avoided. Such removal can be observed when catalytically active substances are applied by means of a binder, such as an adhesive, in which case the catalytic activity of corresponding filters decreases significantly during use.

Furthermore, a disadvantage of applying catalytically active substances by means of a binder to a polymer fibre or a polymeric sheet is that fabrics, knits, or nonwovens produced therefrom often have a significantly increased resistance to air permeability compared to non-finished fabrics, knits, or nonwovens, so that a large pressure loss at the filter occurs during the use of corresponding materials as a filter. This is disadvantageous in terms of fluid dynamics and therefore usually undesirable because overcoming the pressure difference expends energy. In contrast, the polymer fibres or polymeric sheets finished according to the disclosure and fabrics, knits, or nonwovens produced therefrom show a significantly smaller pressure loss and are thus favored in terms of fluid dynamics.

The specific surface area of a textile is directly dependent on the fibre diameter. While current commercially available hose filters predominantly use needle-punched nonwovens made of PET with a titre (linear density) of about 5 dtex, there are also hose filters of a newer generation with significantly lower titres, e.g. based on microfilament nonwovens (see DE102007023806). The technical advantage of these newer hose filters is that good separation performance can be achieved with a comparatively much lower area weight, as the pore-forming filaments have smaller diameters than conventional fibres, and because they are not mechanically needled, which leads to damage and thus to a deterioration of their mechanical properties, but are hydroentangled. At the same time, microfilaments of the “segmented pie” type have a comparatively much larger surface area, as shown in the following example calculation.

The surface area per 100 g of nonwoven is calculated as:
Case 1, 5 dtex, PET: 13.5 m²
Case 2, 2.4 dtex PIE 16 with 70% PET and 30% PA6: for PET 65 m², for PA6 57 m²

That is, with the same area weight, namely 100 g (per m²), the needle-punched nonwoven has 13.5 m² of PET surface per 100 g, while the spunbond fabric, segmented PIE 70/30 at 2.4 dtex before splitting, has about 5 times the surface of PET after splitting by hydroentanglement.

### DRAWINGS

The drawing described herein is for illustrative purposes only of selected embodiment(s) and not all possible implementations, and are not intended to limit the scope of the present disclosure.

FIG. 1 shows a scanning electron microscopic image of the cross-section of a polymer fibre finished according to the disclosure.

Corresponding reference numerals indicate corresponding parts throughout the view of the drawing.

### DETAILED DESCRIPTION

Example embodiment(s) will now be described more fully with reference to the accompanying drawing.

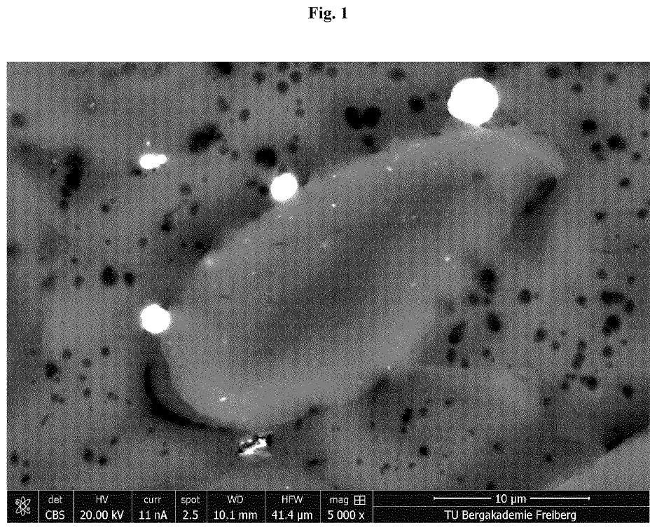

The bright points depicted in FIG. 1 show TiO₂ particles with a diameter of 2 μm to 4 μm. The particles were impregnated with a platinum-containing solution, so that catalytically active platinum atoms or agglomerates are located on the surface of the TiO₂ particles. Here, the TiO₂ particles partially penetrate into the fibre structure and are thus firmly bonded to the fibre surface. The fibre diameter remains almost unchanged compared to a non-finished fibre, which means that the hydraulic resistance of a fabric, knit, or nonwoven made from a correspondingly finished fibre also remains almost unchanged compared to a non-finished fabric, knit, or nonwoven. The same applies to fabrics, knits, or nonwovens that are subsequently finished according to the disclosure. These also exhibit almost unchanged resistance to air permeability compared to comparable non-finished fabrics, knits, or nonwovens.

The disclosure is explained in more detail below using exemplary embodiments.

### Example 1

Four 10 g samples of a polyester fabric (PET needle-punched nonwoven with around 5 dtex and 550 g/m²) were individually contacted with a dispersion bath, which contained 1.5 wt. %, 2.0 wt. %, 3.0 wt. %, or 4.0 wt. % platinum-impregnated TiO₂ particles, respectively. To each dispersion bath, 0.3 g of a dispersing agent (CHT Dispergator SMS) was added and the pH value of the dispersion baths was adjusted to pH 4.5 using diluted acetic acid. The dispersion baths were topped up to 100 mL with de-hardened water (soft water) and were homogenized. Subsequently, the fabric samples, together with the respective dispersion baths, were heated to 135° C. in dye bombs (autoclaves) under mechanical movement in a glycol bath with a heating rate of 3° C./min and then held at this temperature for 60 min. This was followed by cooling to 60° C. with a cooling rate of 6° C./min. Before opening the autoclaves, they were cooled to room temperature with running water. The fabric samples were removed and each was washed with 2 L of softened water for 1 m. Subsequently, the samples were dried by spinning for 1 min. After a final drying of the samples at 40° C. in a drying cabinet, the loading of the respective samples was determined by weighing and comparing to the sample weight before treatment. The following loadings were determined:

| | | | | | | Air |
|---|---|---|---|---|---|---|
| | | | | | | permeability |
| | | Pressure | | | | (rel. value |
| | | pulse | | Activity/ | | according to |
| Treatment | Type of | test | Loading | 50% CO | | DIN EN ISO |
| of material | catalyst | (6 bar) | weight | conversion | | 9237)/L/m³/s |
| untreated | none | none | 0 | n/a | | 325.2 |
| polyester | | | | | | |
| 550 g/m² | | | | | | |
| 15% Disp | Pt/TiO₂ | none | 0.83 | 100° | C. | 318.4 |
| | | 500 | no | 105° | C. | 320.8 |
| | | | detectable | | | |
| | | | change | | | |
| | | 1000 | no | 105° | C. | 321.2 |
| | | | detectable | | | |
| | | | change | | | |
| 20% Disp | Pt/TiO₂ | none | 1.34 | 99° | C. | 290.4 |
| | | 500 | no | 100° | C. | 296.2 |
| | | | detectable | | | |
| | | | change | | | |
| | | 1000 | no | 110° | C. | 293 |
| | | | detectable | | | |
| | | | change | | | |
| 30% Disp | Pt/TiO₂ | none | 1 | 90° | C. | 313.2 |
| | | 500 | no | 91° | C. | 317 |
| | | | detectable | | | |
| | | | change | | | |
| | | 1000 | no | 92° | C. | 318.4 |
| | | | detectable | | | |
| | | | change | | | |
| 40% Disp | Pt/TiO₂ | none | 1.58 | 85° | C. | 306 |
| | | 500 | no | 91° | C. | 312.8 |
| | | | detectable | | | |
| | | | change | | | |
| | | 1000 | no | 92° | C. | 312.4 |
| | | | detectable | | | |
| | | | change | | | |

### Example 2

A 10 g sample of a PET/PA6 microfilament nonwoven with an area weight of 80 g/m² made of PET/PA6 with a volumetric ratio of around 70/30 and a titre after splitting of about 0.2 dtex for PET and about 0.1 dtex for PA6 was treated analogously to Example 1 and contacted with a dispersion bath, which contained 4 wt. % platinum-impregnated TiO₂ particles. A loading of 3.05 g was determined, which corresponds to about twice the loading in comparison, of which 0.2 g was detached after 500 pressure pulse tests and a further 0.08 g after 1000 pressure pulses. The fact that not 5 times the amount of Pt/TiO₂ was incorporated can be explained by the fact that, in the spunbond process, a comparatively strong stretching leads to higher crystallinity of the polymer filaments and accordingly fewer amorphous areas are available for the fixation of the catalyst particles. Also, the fixation to polyamide is not quite as stable as with PET. The fact that a portion of the deposited catalyst particles can be detached by the compressed air pulses may be explained by the fact that some of the particles are filtered out due to the smaller pore size of the microfilament material, but are not fixed. With higher area weights when using more layers (tortuosity) and increased density after the hydroentanglement, the filtering out of the catalyst particles becomes more significant. However, it could be shown that it is also fundamentally possible to achieve such stronger stretched microfilament nonwovens with dispersion fixation.

The foregoing description of the embodiment(s) has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for surface-catalytically finishing polymer fibres and/or polymeric sheets, said method comprising the steps:
a) providing a polymer fibre or a polymeric sheet made of an amorphous or partially amorphous polymer;
b) providing an aqueous dispersion of a catalyst support particle;
c) contacting the polymer fibre or the polymeric sheet with the aqueous dispersion of the catalyst support particle,
wherein the catalyst support particle has a particle size in a range of between ≥0.5 μm and ≤20 μm, preferably between ≥1 μm and ≤10 μm, in particular between ≥1.5 μm and ≤5 μm, and in that the contacting in step c) takes place at a temperature in a range of between ≥15° C. and ≤60° C., preferably between ≥20° C. and ≤50° C., in particular between ≥30° C. and ≤40° C. above the glass transition temperature T_{G} of the polymer, and wherein the catalyst support particle is impregnated with a catalytically active metal or metal oxide.

2. The method according to claim 1, wherein the polymer fibre or the polymeric sheet is contacted at the specified temperature for a period of between ≥30 min and ≤360 min, preferably between ≥60 min and ≤240 min, in particular between ≥90 min and ≤120 min.

3. The method according to claim 1, wherein the catalyst support particle is porous and preferably has a porosity Φ in a range of between ≥10% and ≤60%.

4. The method according to claim 3, wherein the catalyst support particle is selected from the group consisting of titanium dioxide, cordierite, zeolites, magnesium silicate, or layered silicate.

5. The method according to claim 1, wherein the catalytically active metal is at least one metal selected from the group consisting of metals from the group of outer transition metals of the periodic table, preferably platinum, nickel, gold, silver, rhenium, cobalt, vanadium, chromium, copper, palladium, iridium, rhodium, and zirconium, or an oxide or a mixed oxide thereof.

6. The method according to claim 1, wherein the polymer is selected from the group consisting of polyethylene terephthalate (PET), polybutylene terephthalate (PBT), polylactic acid (PLA), polytrimethylene terephthalate (PTT), polyethylene naphthalate (PEN), polycarbonate (PC), polyamide (PA), polyarylate (PAR), polyester carbonate (PEC), or copolymers or blends thereof.

7. The method according to claim 1, wherein the aqueous dispersion is adjusted to a pH value in a range of between ≥pH 1 and ≤pH 5.

8. A surface-catalytically finished polymer fibre or surface-catalytically finished polymeric sheet, wherein the fibre or the sheet has redox-active catalyst support particles on its surface, wherein the catalyst support particles include at least one metal from the group of outer transition metals of the periodic table, preferably platinum, nickel, gold, silver, rhenium, cobalt, vanadium, chromium, copper, palladium, iridium, rhodium, and zirconium, or an oxide or a mixed oxide thereof, and wherein the catalyst support particles have at least partially penetrated into the polymer fibre or the polymeric sheet and are thus bonded to the polymer fibre or the polymeric sheet without any additional fastening means.

9. The surface-catalytically finished polyester fibre or surface-catalytically finished polyester sheet according to claim 8, wherein the catalyst support particles are immobilized on the surface of the fibre or the sheet in a mechanically stable manner, and at least ≥500 compressed air pulses with an overpressure of 6 bar, each lasting 1 second, lead to detachments of the catalytic particles of ≤5% based on the total mass of the catalytic particles.

10. A method for producing a filter for particle separation in an air purification system comprising a surface-catalytically finished polymer fibre and/or a surface-catalytically finished polymeric sheet.
